(19) United States
(12) Patent Application Publication
(10) Pub. No.: US 2021/0100847 A1
(43) Pub. Date: Apr. 8, 2021

(54) STEM CELLS FOR THE TREATMENT OF CHRONIC TRAUMATIC ENCEPHALOPATHY

(71) Applicant: WCTE Inc., Pinole, CA (US)

(72) Inventors: Thomas Ichim, San Diego, CA (US); Wes Chandler, Pinole, CA (US)

(73) Assignee: WCTE Inc., Pinole, CA (US)

(21) Appl. No.: 17/063,421

(22) Filed: Oct. 5, 2020

Related U.S. Application Data
(60) Provisional application No. 62/910,308, filed on Oct. 3, 2019.

(51) Int. Cl. A61K 35/28; A61K 35/545; A61K 35/15

(52) U.S. Cl. CPC A61K 35/28; A61K 35/15; A61K 35/545

(57) ABSTRACT

Disclosed are new, useful and non-obvious methodologies and compositions of matter for the treatment of chronic traumatic encephalopathy (CTE) using stem cells and cells possessing stem cell-like activity. The treatment of CTE is accomplished by utilizing stem cells to: a) reduce oxidative stress; b) suppress inflammation; c) enhance neurogenesis; and d) stimulate axonal regrowth. In one embodiment said stem cells are mesenchymal stem cells derived from umbilical cord and other perinatal tissues. In another embodiment, said stem cells are bone marrow derived. In yet another embodiment said stem cells are adipose derived. In one embodiment, products derived from said regenerative cells are comprised of cellular lysate, apoptotic bodies, exosomes, and other microvesicles. In one embodiment, said regenerative cells and/or said products derived from said regenerative cells are administered subsequent to one ore multiple head injuries. In other embodiments said products are administered in combination with neurorestorative and/or neuroprotective interventions.

### CROSS-REFERENCE TO RELATED APPLICATIONS

This application claims the benefit of priority to U.S. Provisional Application No. 62/910,308, filed Oct. 3, 2019, the contents of which are incorporated herein by reference.

### FIELD OF THE INVENTION

The Invention pertains to the area of head trauma, more particularly the invention pertains to the field of chronic traumatic encephalopathy (CTE), more particularly, the invention pertains to the field of regenerative medicine for treatment of CTE.

### BACKGROUND OF THE INVENTION

Chronic Traumatic Encephalopathy (CTE) has been originally described in the sport of boxing under the name “punch drunk syndrome”. It is known that professional boxers with multiple bouts and repeated head blows are prone to chronic traumatic encephalopathy (CTE). Repeated head blows produce rotational acceleration of the brain, diffuse axonal injury, and other neuropathological features. CTE includes motor changes such as tremor, dysarthria, and parkinsonism; cognitive changes such as mental slowing and memory deficits; and psychiatric changes such as explosive behavior, morbid jealousy, pathological intoxication, and [1-6].

It is believed that in England at least 17% of boxers have CTE disturbed gait and coordination, slurred speech and tremors, as well as cerebral dysfunction causing cognitive impairments and neurobehavioural disturbances [7]. In one study, diffusion tensor imaging (DTI), which is sensitive to microscopic white matter changes when routine MR imaging is unrevealing [8, 9], was used together with tract-based spatial statistics (TBSS) together with neuropsychological examination of executive functions and memory to investigate a collective of 31 male amateur boxers and 31 age-matched controls as well as a subgroup of 19 individuals, respectively, who were additionally matched for intellectual performance (IQ). It was found that participants had normal findings in neurological examination and conventional MR. Amateur boxers did not show deficits in neuropsychological tests when their IQ was taken into account. Fractional anisotropy was significantly reduced, while diffusivity measures were increased along central white matter tracts in the boxers group. These changes were in part associated with the number of fights. This study demonstrated that TBSS revealed widespread white matter disturbance partially related to the individual fighting history in amateur boxers. These findings closely resemble those in patients with accidental TBI and indicate similar histological changes in amateur boxers [10].

In addition to boxing, Jockeys have also been reported to suffer from CTE, in a 1976 publication, Foster et al reported Five National Hunt jockeys have been found to have post-traumatic encephalopathy-three with epilepsy and two with significant intellectual and psychological deterioration [11]. Other reports of jockey's having similar situations have been described [12]. Numerous other causes of CTE have been described including whiplash [13], shaken baby syndrome [14], wrestling [15], military combat [16, 17], football [18-22], rugby [23], soccer [24, 25], jail head trauma [26], shotgun injury [27], mixed martial arts [28].

One study in the journal JAMA examined a case series of 202 football players whose brains were donated for research. Neuropathological evaluations and retrospective telephone clinical assessments (including head trauma history) with informants were performed blinded. Online questionnaires ascertained athletic and military history. Neuropathological diagnoses of neurodegenerative diseases, including CTE, based on defined diagnostic criteria; CTE neuropathological severity (stages Ito IV or dichotomized into mild [stages I and II] and severe [stages III and IV]); informant-reported athletic history and, for players who died in 2014 or later, clinical presentation, including behavior, mood, and cognitive symptoms and dementia. Among 202 deceased former football players (median age at death, 66 years [interquartile range, 47-76 years]), CTE was neuropathologically diagnosed in 177 players (87%; median age at death, 67 years [interquartile range, 52-77 years]; mean years of football participation, 15.1 [SD, 5.2]), including 0 of 2 pre-high school, 3 of 14 high school (21%), 48 of 53 college (91%), 9 of 14 semiprofessional (64%), 7 of 8 Canadian Football League (88%), and 110 of 111 National Football League (99%) players. Neuropathological severity of CTE was distributed across the highest level of play, with all 3 former high school players having mild pathology and the majority of former college (27 [56%]), semiprofessional (5 [56%]), and professional (101 [86%]) players having severe pathology. Among 27 participants with mild CTE pathology, 26 (96%) had behavioral or mood symptoms or both, 23 (85%) had cognitive symptoms, and 9 (33%) had signs of dementia. Among 84 participants with severe CTE pathology, 75 (89%) had behavioral or mood symptoms or both, 80 (95%) had cognitive symptoms, and 71 (85%) had signs of dementia. In a convenience sample of deceased football players who donated their brains for research, a high proportion had neuropathological evidence of CTE, suggesting that CTE may be related to prior participation in football [29].

In another study the authors examined the effect of age of first exposure to tackle football on chronic traumatic encephalopathy (CTE) pathological severity and age of neurobehavioral symptom onset in tackle football players with neuropathologically confirmed CTE. The sample included 246 tackle football players who donated their brains for neuropathological examination. Two hundred eleven were diagnosed with CTE (126 of 211 were without comorbid neurodegenerative diseases), and 35 were without CTE. Informant interviews ascertained age of first exposure and age of cognitive and behavioral/mood symptom onset. Analyses accounted for decade and duration of play. Age of exposure was not associated with CTE pathological severity, or Alzheimer's disease or Lewy body pathology. In the 211 participants with CTE, every 1 year younger participants began to play tackle football predicted earlier reported cognitive symptom onset by 2.44 years (p<0.0001) and behavioral/mood symptoms by 2.50 years (p<0.0001). Age of exposure before 12 predicted earlier cognitive (p<0.0001) and behavioral/mood (p<0.0001) symptom onset by 13.39 and 13.28 years, respectively. In participants with dementia, younger age of exposure corresponded to earlier functional impairment onset. Similar effects were observed in the 126 CTE-only participants. Effect sizes were comparable in participants without CTE. In this sample of deceased tackle football players, younger age of exposure to tackle football was not associated with CTE pathological severity, but predicted earlier neurobehavioral symptom onset. Youth exposure to tackle football may reduce resiliency to late-life neuropathology [30].

### SUMMARY

Methods herein are directed to methods of treating chronic traumatic encephalopathy comprising the steps of:
a) identifying a patient with elevated inflammatory markers subsequent to a head injury;
b) administering a therapeutically sufficient amount of regenerative cells to said patient; and
c) administering a therapeutically sufficient amount of products derived from said regenerative cells.

Further embodiments include methods wherein said inflammatory markers assessed subsequent to said head injury are selected from the group consisting of: a) C-reactive protein; b) interleukin-1; c) interleukin-6; d) interleukin-8; e) interleukin-33; f) erythrocyte sedimentation ratio; g) TNF-alpha; and h) interferon-gamma.

Further embodiments include methods wherein elevated means a concentration at least 20% higher as compared to standard laboratory values.

Further embodiments include methods wherein said regenerative cells are monocytes.

Further embodiments include methods wherein said regenerative cells are pluripotent stem cells.

Further embodiments include methods wherein said pluripotent stem cells are selected from the group consisting of: a) acid stimulated retrodifferentiated stem cells; b) parthenogenic derived stem cells; c) inducible pluripotent stem cells; d) somatic cell nuclear transfer derived stem cells; e) cytoplasmic transfer derived stem cells; and f) stimulus-triggered acquisition of pluripotency.

Further embodiments include methods wherein said regenerative cells are hematopoietic stem cells.

Further embodiments include methods wherein said hematopoietic stem cells are capable of multi-lineage reconstitution in an immunodeficient host.

Further embodiments include methods wherein said hematopoietic stem cells express the c-kit protein.

Further embodiments include methods wherein said hematopoietic stem cells express the Sca-1 protein.

Further embodiments include methods wherein said hematopoietic stem cells express CD34.

Further embodiments include methods wherein said hematopoietic stem cells express CD133.

Further embodiments include methods wherein said hematopoietic stem cells lack expression of lineage markers.

Further embodiments include methods wherein said hematopoietic stem cells lack expression of CD38.

Further embodiments include methods wherein said hematopoietic stem cells are positive for expression of c-kit and Sca-1 and substantially lack expression of lineage markers.

Further embodiments include methods wherein said hematopoietic stem cells are derived from a group of sources, said group comprising of: a) peripheral blood; b) mobilized peripheral blood; c) bone marrow; d) cord blood; e) adipose stromal vascular fraction; and f) derived from progenitor cells.

Further embodiments include methods wherein said progenitor cell expresses CD56.

Further embodiments include methods wherein said regenerative cells are mesenchymal stem cells.

Further embodiments include methods wherein said mesenchymal stem cells are plastic adherent.

Further embodiments include methods wherein said mesenchymal stem cells express a marker selected from a group comprising of: a) CD73; b) CD90; and c) CD105.

Further embodiments include methods wherein said mesenchymal stem cells lack expression of a marker selected from a group comprising of: a) CD14; b) CD45; and c) CD34.

Further embodiments include methods wherein said mesenchymal stem cells are derived from tissues selected from a group comprising of: a) bone marrow; b) peripheral blood; c) adipose tissue; d) mobilized peripheral blood; e) umbilical cord blood; f) Wharton's jelly; g) umbilical cord tissue; h) skeletal muscle tissue; i) subepithelial umbilical cord; j) endometrial tissue; k) menstrual blood; and l) fallopian tube tissue.

Further embodiments include methods wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Further embodiments include methods wherein said mesenchymal stem cells from umbilical cord tissue do not express markers selected from a group comprising of: a) CD117; b) CD31; c) CD34; and CD45;

Further embodiments include methods wherein said mesenchymal stem cells from umbilical cord tissue express, relative to a human fibroblast, increased levels of interleukin 8 and reticulon 1

Further embodiments include methods wherein said mesenchymal stem cells from umbilical cord tissue have the potential to differentiate into cells of at least a skeletal muscle, vascular smooth muscle, pericyte or vascular endothelium phenotype.

Further embodiments include methods wherein said mesenchymal stem cells from umbilical cord tissue express markers selected from a group comprising of: a) CD10; b) CD13; c) CD44; d) CD73; and e) CD90.

Further embodiments include methods wherein said umbilical cord tissue mesenchymal stem cell is an isolated umbilical cord tissue cell isolated from umbilical cord tissue substantially free of blood that is capable of self-renewal and expansion in culture,

Further embodiments include methods wherein said umbilical cord tissue mesenchymal stem cells has the potential to differentiate into cells of other phenotypes.

Further embodiments include methods wherein said other phenotypes comprise: a) osteocytic; b) adipogenic; and c) chondrogenic differentiation.

Further embodiments include methods wherein said cord tissue derived mesenchymal stem cells can undergo at least 20 doublings in culture.

Further embodiments include methods wherein said cord tissue derived mesenchymal stem cell maintains a normal karyotype upon passaging

Further embodiments include methods wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Further embodiments include methods wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Further embodiments include methods wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1

Further embodiments include methods wherein said umbilical cord tissue derived cells express markers selected from a group comprising of: a) TRA1-60; b) TRA1-81; c) SSEA3; d) SSEA4; and e) NANOG.

Further embodiments include methods wherein said umbilical cord tissue-derived cells are positive for alkaline phosphatase staining.

Further embodiments include methods wherein said umbilical cord tissue-derived cells are capable of differentiating into one or more lineages selected from a group comprising of; a) ectoderm; b) mesoderm, and; c) endoderm.

Further embodiments include methods wherein said bone marrow derived mesenchymal stem cells possess markers selected from a group comprising of: a) CD73; b) CD90; and c) CD105.

Further embodiments include methods wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.

Further embodiments include methods wherein said bone marrow derived mesenchymal stem cell is a mesenchymal stem cell progenitor cell.

Further embodiments include methods wherein said mesenchymal progenitor cells are a population of bone marrow mesenchymal stem cells enriched for cells containing STRO-1

Further embodiments include methods wherein said mesenchymal progenitor cells express both STRO-1 and VCAM-1.

Further embodiments include methods wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Further embodiments include methods wherein said bone marrow mesenchymal stem cells lack expression of CD14, CD34, and CD45.

Further embodiments include methods wherein said STRO-1 expressing cells are positive for a marker selected from a group comprising of: a) VACM-1; b) TKY-1; c) CD146 and; d) STRO-2

Further embodiments include methods wherein said bone marrow mesenchymal stem cell express markers selected from a group comprising of: a) CD13; b) CD34; c) CD56 and; d) CD117

Further embodiments include methods wherein said bone marrow mesenchymal stem cells do not express CD10.

Further embodiments include methods wherein said bone marrow mesenchymal stem cells do not express CD2, CD5, CD14, CD19, CD33, CD45, and DRII.

Further embodiments include methods wherein said bone marrow mesenchymal stem cells express CD13,CD34, CD56, CD90, CD117 and nestin, and which do not express CD2, CD3, CD10, CD14, CD16, CD31, CD33, CD45 and CD64.

Further embodiments include methods wherein said skeletal muscle stem cells express markers selected from a group comprising of: a) CD13; b) CD34; c) CD56 and; d) CD117

Further embodiments include methods wherein said skeletal muscle mesenchymal stem cells do not express CD10.

Further embodiments include methods wherein said skeletal muscle mesenchymal stem cells do not express CD2, CD5, CD14, CD19, CD33, CD45, and DRII.

Further embodiments include methods wherein said bone marrow mesenchymal stem cells express CD13,CD34, CD56, CD90, CD117 and nestin, and which do not express CD2, CD3, CD10, CD14, CD16, CD31, CD33, CD45 and CD64.

Further embodiments include methods wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Further embodiments include methods wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.

Further embodiments include methods wherein said subepithelial umbilical cord derived mesenchymal stem cells express CD29, CD73, CD90, CD166, SSEA4, CD9, CD44, CD146, and CD105.

Further embodiments include methods wherein said subepithelial umbilical cord derived mesenchymal stem cells do not express CD45, CD34, CD14, CD79, CD106, CD86, CD80, CD19, CD117, Stro-1, and HLA-DR.

Further embodiments include methods wherein said subepithelial umbilical cord derived mesenchymal stem cells are positive for SOX2.

Further embodiments include methods wherein said subepithelial umbilical cord derived mesenchymal stem cells are positive for OCT4.

Further embodiments include methods wherein said subepithelial umbilical cord derived mesenchymal stem cells are positive for OCT4 and SOX2.

Further embodiments include methods wherein said regenerative cell derived products is stem cell conditioned media.

Further embodiments include methods wherein said regenerative cell derived products are stem cell derived microvesicles.

Further embodiments include methods wherein said regenerative cell derived products are stem cell derived exosomes.

Further embodiments include methods wherein said regenerative cell derived products are stem cell derived apoptotic vesicles.

Further embodiments include methods wherein said regenerative cell derived products are stem cell derived miRNAs.

Further embodiments include methods wherein said exosomes possess a size of between 30 nm and 150 nm as determined by electron microscopy.

Further embodiments include methods wherein said exosome possesses a size of between 2 nm and 200 nm, as determined by filtration against a 0.2 .mu.M filter and concentration against a membrane with a molecular weight cut-off of 10 kDa, or a hydrodynamic radius of below 100 nm as determined by laser diffraction or dynamic light scattering.

Further embodiments include methods wherein said exosome possesses a lipid selected from the group consisting of: a) phospholipids; b) phosphatidyl serine; c) phosphatidyl inositol; d) phosphatidyl choline; e) sphingomyelin; f) ceramides; g) glycolipid; h) cerebroside; i) steroids, and j) cholesterol.

Further embodiments include methods wherein said exosome possesses a lipid raft.

Further embodiments include methods wherein said exosome expresses antigenic markers on surface of said exosome, wherein said antigenic markers are selected from a group comprising of: a) CD9; b) CD63; c) CD81; d) ANXA2; e) ENO1; f) HSP9OAA1; g) EEF1A1; h) YWHAE; i) SDCBP; j) PDCD6IP; k) ALB; l) YWHAZ; m) EEF2; n) ACTG1; o) LDHA; p) HSP90AB1; q) ALDOA; r) MSN; s) ANXA5; t) PGK1; and u) CFL1.

Further embodiments include methods wherein administration of regenerative cells and products thereof is performed in a patient suffering from CTE.

### DESCRIPTION OF INVENTION

The invention discloses the novel, useful, and unexpected finding that administration of stem cells induces regeneration/protection/prophylaxis in patients suffering from CTE. More specifically, the invention provides means of using stem cells, and/or products derived from said stem cells to inhibit the progressive neuronal loss, cognitive loss, and microglial activation as a result of CTE. In one embodiment the invention provides the administration of mesenchymal stem cells as a means of enhancing function of neurons, and/or protecting neurons in a patient with CTE.

For the practice of the invention, a preferred embodiment is the administration of mesenchymal stem cells (MSC) intravenously at concentrations sufficient to prevent CTE and or reverse CTE.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably.

Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

Said MSC may be expanded and utilized by administration themselves, or may be cultured in a growth media in order to obtain conditioned media, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

Mesenchymal stem cells (“MSC”) were originally derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2.sup.+SH4.sup.+CD29.sup.+CD44.sup.+CD71.sup.+CD90.sup.+CD106.sup.+CD120a.sup.+CD124.sup.+CD14.sup.−CD34.sup.- CD45.sup.−)). The invention teaches the use of various mesenchymal stem cells

In one embodiment MSC donor lots are generated from umbilical cord tissue. Means of generating umbilical cord tissue MSC have been previously published and are incorporated by reference [31-37]. The term “umbilical tissue derived cells (UTC)” refers, for example, to cells as described in U.S. Pat. Nos. 7,510,873, 7,413,734, 7,524,489, and 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36kDa; Homo sapiens cDNA FU12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FU20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FU14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit Vila polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes.

Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue. In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest. While the use of enzyme is presently preferred, it is not required for isolation methods as provided herein. Methods based on mechanical separation alone may be successful in isolating the instant cells from the umbilicus as discussed above. The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells. Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA , washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In one embodiment, hematopoietic stem cells are CD34+ cells isolated from the peripheral blood, bone marrow, or umbilical cord blood. Specifically, the hematopoietic stem cells may be derived from the blood system of mammalian animals, include but not limited to human, mouse, rat, and these hematopoietic stem cells may be harvested by isolating from the blood or tissue organs in mammalian animals. Hematopoietic stem cells may be harvested from a donor by any known methods in the art. For example, U.S. Pub. 2013/0149286 details procedures for obtaining and purifying stem cells from mammalian cadavers. Stem cells may be harvested from a human by bone marrow harvest or peripheral blood stem cell harvest, both of which are well known techniques in the art. After stem cells have been obtained from the source, such as from certain tissues of the donor, they may be cultured using stem cell expansion techniques. Stem cell expansion techniques are disclosed in U.S. Pat. No. 6,326,198 to Emerson et al., entitled “Methods and compositions for the ex vivo replication of stem cells, for the optimization of hematopoietic progenitor cell cultures, and for increasing the metabolism, GM-CSF secretion and/or IL-6 secretion of human stromal cells,” issued Dec. 4, 2001; U.S. Pat. No. 6,338,942 to Kraus et al., entitled “Selective expansion of target cell populations,” issued Jan. 15, 2002; and U.S. Pat. No. 6,335,195 to Rodgers et al., entitled “Method for promoting hematopoietic and cell proliferation and differentiation,” issued Jan. 1, 2002, which are hereby incorporated by reference in their entireties. In some embodiments, stem cells obtained from the donor are cultured in order to expand the population of stem cells. In other preferred embodiments, stem cells collected from donor sources are not expanded using such techniques. Standard methods can be used to cyropreserve the stem cells.

In some embodiments of the invention, where there are risks associated with particular types of stem cells, for example, pluripotent stem cells, said stem cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of stem cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of stem cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of stem cells. Certain embodiments incorporate stem cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, stem cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

In one embodiment of the invention, exosomes are purified from mesenchymal stem cells by obtaining a mesenchymal stem cell conditioned medium, concentrating the mesenchymal stem cell conditioned medium, subjecting the concentrated mesenchymal stem cell conditioned medium to size exclusion chromatography, selecting UV absorbent fractions at 220 nm, and concentrating fractions containing exosomes.

Exosomes, also referred to as “particles” may comprise vesicles or a flattened sphere limited by a lipid bilayer. The particles may comprise diameters of 40-100 nm. The particles may be formed by inward budding of the endosomal membrane. The particles may have a density of .about.1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in mesenchymal stem cells or mesenchymal stem cell conditioned medium (MSC-CM), such as a protein characteristic or specific to the MSC or MSC-CM. They may comprise RNA, for example miRNA. Said particles may possess one or more genes or gene products found in MSCs or medium which is conditioned by culture of MSCs. The particle may comprise molecules secreted by the MSC. Such a particle, and combinations of any of the molecules comprised therein, including in particular proteins or polypeptides, may be used to supplement the activity of, or in place of, the MSCs or medium conditioned by the MSCs for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. The particle may be used for any of the therapeutic purposes that the MSC or MSC-CM may be put to use.

In one embodiment, MSC exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

In one embodiment of the invention, stem cells, and/or stem cell derivatives, such as exosomes are administered to patients with CTE.

### REFERENCES

1. Zhuo, Y., et al., White matter impairment in type 2 diabetes mellitus with and without microvascular disease. Neuroimage Clin, 2019. 24: p. 101945.
2. Mendez, M. F., The neuropsychiatric aspects of boxing. Int J Psychiatry Med, 1995. 25(3): p. 249-62.
3. Jordan, B. D., Neurologic aspects of boxing. Arch Neurol, 1987. 44(4): p. 453-9.
4. McKee, A. C., et al., Chronic traumatic encephalopathy in athletes: progressive tauopathy after repetitive head injury. J Neuropathol Exp Neurol, 2009. 68(7): p. 709-35.
5. Gavett, B. E., et al., Clinical appraisal of chronic traumatic encephalopathy: current perspectives and future directions. Curr Opin Neurol, 2011. 24(6): p. 525-31.
6. Stern, R. A., et al., Long-term consequences of repetitive brain trauma: chronic traumatic encephalopathy. PM R, 2011. 3(10 Suppl 2): p. S460-7.
7. McCrory, P., T. Zazryn, and P. Cameron, The evidence for chronic traumatic encephalopathy in boxing. Sports Med, 2007. 37(6): p. 467-76.
8. Bouziane, C., et al., White Matter by Diffusion MRI Following Methylphenidate Treatment: A Randomized Control Trial in Males with Attention-Deficit/Hyperactivity Disorder. Radiology, 2019: p. 182528.
9. Kochunov, P., et al., White Matter in Schizophrenia Treatment Resistance. Am J Psychiatry, 2019: p. appiajp201918101212.
10. Herweh, C., et al., Reduced white matter integrity in amateur boxers. Neuroradiology, 2016. 58(9): p. 911-20.
11. Foster, J. B., R. Leiguarda, and P. J. Tilley, Brain damage in National Hunt jockeys. Lancet, 1976. 1(7967): p. 981-3.
12. McCrory, P., M. Turner, and J. Murray, A punch drunk jockey? Br J Sports Med, 2004. 38(3): p. e3.
13. Berstad, J. R., et al., Whiplash: chronic organic brain syndrome without hydrocephalus ex vacuo. Acta Neurol Scand, 1975. 51(4): p. 268-84.
14. Squier, W., Shaken baby syndrome: the quest for evidence. Dev Med Child Neurol, 2008. 50(1): p. 10-4.
15. Omalu, B. I., et al., Chronic traumatic encephalopathy in a professional American wrestler. J Forensic Nurs, 2010. 6(3): p. 130-6.
16. Omalu, B., et al., Chronic traumatic encephalopathy in an Iraqi war veteran with posttraumatic stress disorder who committed suicide. Neurosurg Focus, 2011. 31(5): p. E3.
17. Hasoon, J., Blast-associated traumatic brain injury in the military as a potential trigger for dementia and chronic traumatic encephalopathy. US Army Med Dep J, 2017(1-17): p. 102-105.
18. Omalu, B. I., et al., Chronic traumatic encephalopathy in a National Football League player. Neurosurgery, 2005. 57(1): p. 128-34; discussion 128-34.
19. Omalu, B. I., et al., Chronic traumatic encephalopathy in a national football league player: part II. Neurosurgery, 2006. 59(5): p. 1086-92; discussion 1092-3.
20. Breedlove, E. L., et al., Biomechanical correlates of symptomatic and asymptomatic neurophysiological impairment in high school football. J Biomech, 2012. 45(7): p. 1265-72.
21. Maroon, J. C., et al., Chronic traumatic encephalopathy in contact sports: a systematic review of all reported pathological cases. PLoS One, 2015. 10(2): p. e0117338.
22. Bieniek, K. F., et al., Association between contact sports participation and chronic traumatic encephalopathy: a retrospective cohort study. Brain Pathol, 2019.
23. McMillan, T. M., et al., Long-term health outcomes after exposure to repeated concussion in elite level: rugby union players. J Neurol Neurosurg Psychiatry, 2017. 88(6): p. 505-511.
24. Ling, H., et al., Mixed pathologies including chronic traumatic encephalopathy account for dementia in retired association football (soccer) players. Acta Neuropathol, 2017. 133(3): p. 337-352.
25. Nitrini, R., Soccer (Football Association) and chronic traumatic encephalopathy: A short review and recommendation. Dement Neuropsychol, 2017. 11(3): p. 218-220.
26. Siegler, A., et al., Head Trauma in Jail and Implications for Chronic Traumatic Encephalopathy in the United States: Case Report and Results of Injury Surveillance in NYC Jails. J Health Care Poor Underserved, 2017. 28(3): p. 1042-1049.
27. Tribett, T., et al., Chronic Traumatic Encephalopathy Pathology After Shotgun Injury to the Brain. J Forensic Sci, 2019. 64(4): p. 1248-1252.
28. Lim, L. J. H., R. C. M. Ho, and C. S. H. Ho, Dangers of Mixed Martial Arts in the Development of Chronic Traumatic Encephalopathy. Int J Environ Res Public Health, 2019. 16(2).
29. Mez, J., et al., Clinicopathological Evaluation of Chronic Traumatic Encephalopathy in Players of American Football. JAMA, 2017. 318(4): p. 360-370.
30. Alosco, M .L., et al., Age of first exposure to tackle football and chronic traumatic encephalopathy. Ann Neurol, 2018. 83(5): p. 886-901.
31. Van Pham, P., et al., Isolation and proliferation of umbilical cord tissue derived mesenchymal stem cells for clinical applications. Cell Tissue Bank, 2015.
32. Fazzina, R., et al., A new standardized clinical-grade protocol for banking human umbilical cord tissue cells. Transfusion, 2015. 55(12): p. 2864-73.
33. Bieback, K., Platelet lysate as replacement for fetal bovine serum in mesenchymal stromal cell cultures. Transfus Med Hemother, 2013. 40(5): p. 326-35.
34. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.
35. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.
36. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.
37. Friedman, R., et al., Umbilical cord mesenchymal stem cells: adjuvants for human cell transplantation. Biol Blood Marrow Transplant, 2007. 13(12): p. 1477-86.

1. A method of treating chronic traumatic encephalopathy comprising the steps of:
a) identifying a patient with elevated inflammatory markers subsequent to a head injury; and
b) administering a therapeutically sufficient amount of regenerative cells to said patient.

2. The method of claim 1, wherein said inflammatory markers assessed subsequent to said head injury are selected from the group consisting of: a) C-reactive protein; b) interleukin-1; c) interleukin-6; d) interleukin-8; e) interleukin-33; f) erythrocyte sedimentation ratio; g) TNF-alpha; and h) interferon-gamma.

3. The method of claim 1, wherein elevated means a concentration at least 20% higher as compared to standard laboratory values.

4. The method of claim 1, wherein said regenerative cells are monocytes.

5. The method of claim 1, wherein said regenerative cells are pluripotent stem cells.

6. The method of 1, wherein said regenerative cells are hematopoietic stem cells.

7. The method of claim 6, wherein said hematopoietic stem cells are capable of multi-lineage reconstitution in an immunodeficient host.

8. The method of claim 1, wherein said regenerative cells are mesenchymal stem cells.

9. The method of claim 8, wherein said mesenchymal stem cells are plastic adherent.

10. The method of claim 1, wherein the inflammatory markers are detected in the patient's head or brain.

11. The method of claim 1, wherein the regenerative cells are administered to the patient until the inflammatory markers reduce to standard laboratory values.

12. The method of claim 1, wherein the regenerative cells and products are administered intrathecally.

13. The method of claim 1, further comprising administering a therapeutically sufficient amount of products derived from said regenerative cells.

14. A method of treating chronic traumatic encephalopathy comprising the steps of:
a) identifying a patient suffering a severe head injury;
b) after said head injury; identifying the patient as a high risk of suffering from chronic traumatic encephalopathy; and
c) administering a therapeutically sufficient amount of regenerative cells to said patient.

15. The method of claim 14, wherein identifying the patient as a high risk of suffering from chronic traumatic encephalopathy comprises measuring an abnormal biomarker level from the patient.

16. The method of claim 14, wherein the patient has suffered more than 3 head injuries.
